(19) United States
(12) Patent Application Publication
(10) Pub. No.: US 2026/0200966 A1
(43) Pub. Date: Jul. 16, 2026

(54) CATALYTIC DEPROTECTION METHOD FOR ORGANIC MOLECULES

(71) Applicant: FilmTec Water USA, LLC, Wilmington, DE (US)

(72) Inventors: Martin Deetz, Oxford, PA (US); Jayesh Dharia, Landenberg, PA (US)

(73) Assignee: FilmTec Water USA, LLC, Wilmington, DE (US)

(21) Appl. No.: 19/389,834

(22) Filed: Nov. 14, 2025

Related U.S. Application Data
(60) Provisional application No. 63/720,902, filed on Nov. 15, 2024.

(51) Int. Cl. C07H 21/00; B01J 31/10

(52) U.S. Cl. CPC C07H 21/00; B01J 31/10

(57) ABSTRACT

Organic molecules such as oligonucleotides may be protected, for example with trityl groups or other acid-labile protecting groups that are essential during synthesis but that must be removed to prepare the molecule to undergo a succeeding step in a synthesis or for the molecule, such as an oligonucleotide, to be rendered into a usable form. Accordingly, provided herein is a method for quickly and efficiently deprotecting (e.g., detritylating) organic molecules by contacting a protected molecule, for example a 5′-protected oligonucleotide, with acidic resin particles. The method is particularly advantageous when used in large scale synthesis. The method is quicker than other methods and results in a more streamlined process.

### FIELD OF THE INVENTION

Provided herein is a batch or continuous process for deprotecting dissolved organic molecules, for example oligonucleotides, that are protected with an acid-labile protecting group, for example a trityl group. In particular, a feed stream containing an acid-labile protected organic molecule is contacted with an acid-form cation exchange catalyst, which removes basic impurities (if any) from the feed stream and cleaves the protecting groups from the organic molecules.

### BACKGROUND OF THE INVENTION

Several patents, patent applications and publications are cited in this description in order to more fully describe the state of the art to which this invention pertains. The entire disclosure of each of these patents, patent applications, and publications is incorporated by reference herein.

The use of protecting groups is well-known in organic synthesis. One valuable class of protecting groups is “acid-labile,” i.e., removable under acidic conditions. One particular instance of the powerful utility of acid-labile protecting groups is in oligonucleotide synthesis.

More specifically, RNA/DNA therapeutics is a rapidly emerging field of biotherapeutics. These therapies are based upon a powerful and versatile platform which has the capacity to address many important and as yet unmet clinical needs. These therapeutics are destined to change the standard of care for many diseases. The number of oligonucleotide based drugs under development, and in clinical trials, is growing rapidly with at least 300 such drugs in development globally.

Several methods to synthesize oligonucleotides are described in the literature, including the H-phosphonate and enzymatic processes. General information regarding oligonucleotide synthesis may be found in Gait, M. J. (2017). Oligonucleotide synthesis: methods and applications. CRC Press.

The phosphoramidite chemistry method, which is most commonly used to synthesize oligonucleotides, is also most pertinent to the methods described herein. In phosphoramidite synthesis, phosphorus-based reagents functionalize nucleotides (e.g., A, C, G, T phosphoramidites) and a coupling agent (e.g., a tetrazole derivative) links the functionalized nucleotides together. The coupling agent may also be used to activate the reagent.

In addition, nucleotides may be immobilized on a solid support so that the oligonucleotides are “built up” on the solid support by sequential reaction with additional nucleotides. This method allows for easier purification of the oligonucleotides and reduces the formation and propagation of impurities. Thus, in general, oligonucleotide preparation is conducted by a multistep process such as the following:
a) Synthesis on a solid support
b) Cleavage of the oligonucleotides from the solid support
c) Purification-Solvent and impurity removal
d) Deprotection, e.g., detritylation
e) Optionally, concentration and/or formulation

### a) Synthesis on a Solid Support

The entire targeted sequence is synthesized using solid phase synthesis on crosslinked polystyrene beads or on controlled pore glass (CPG) (described in part by Caruthers, M. H., 1985, Gene synthesis machines: DNA chemistry and its uses. Science, 230(4723), pp. 281-285). The process of deblocking reactive sites and coupling monomers is performed iteratively to eventually provide an oligomeric compound having a predetermined length and base sequence. The synthesis of oligonucleotides using the phosphoramidite chemistry method is outlined in FIG. 1, in which the spheres represent the solid support, B represents a nucleotide base, iPr represents an isopropyl group, DMT represents a dimethoxytrityl group, Py represents pyridine, TCA represents trichloroacetic acid, Ac₂O represents acetic anhydride, and N-Me-Imid represents N-methylimidazole.

The oligonucleotide thus synthesized has acid-labile and base-labile protecting groups on the nucleotide base, phosphate and alcohol groups.

Suitable acid-labile groups include, without limitation, dimethoxytrityl (DMT) and monomethoxytrityl (MMT) groups. As depicted in FIG. 1, the DMT group protects the OH group at the 5′ position on the oligonucleotide. These groups can typically be removed from the oligonucleotides by reaction with acids.

Suitable base-labile groups include, without limitation, acetyl, benzoyl, isobutyryl or phenoxyacetyl groups, esters, and cyanoethyl groups. These groups can typically be removed from the oligonucleotides by reaction with bases.

In addition, the reaction product mixture may also include incomplete oligonucleotides (e.g., due to missing nucleotides or inefficiencies of the synthetic steps).

b) Cleavage of the Oligonucleotide from the Solid Support

After the target oligonucleotide molecule is synthesized, it is cleaved from the solid support using ammonium hydroxide or a primary amine solution as described in Equation I. In addition to breaking the support-oligonucleotide ester bond, this step also removes any base-labile protecting groups that may have been bound to the nucleoside base. The nucleoside base is labelled “base” in Equation 1, and the base-labile protecting groups, if any, are not depicted. The acid labile groups, especially DMT, may be purposefully kept attached to the oligonucleotide during the cleavage step. Due to hydrophobic nature of the DMT group, DMT can aid in purification of the oligonucleotide.

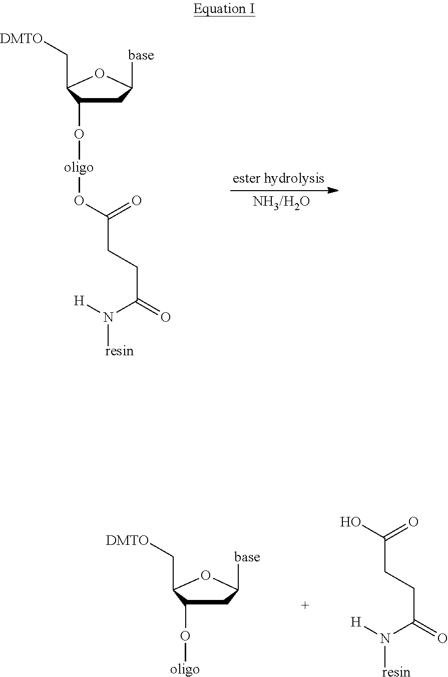

A crude oligonucleotide feed solution is obtained after it is cleaved from solid support post-solid phase synthesis. The crude oligonucleotide feed solution also contains other impurities, such as alkyl amides and organic solvents used during synthesis, or byproducts of the cleavage from the solid support. The crude oligonucleotide solution may further contain incomplete oligonucleotide sequences (also known as shortmers).

### c) Purification—Solvent and Impurity Removal

The crude oligonucleotide solution contains a large amount of ammonia or other free base, as the protected oligonucleotides are normally cleaved from the solid synthesis support using a 30 wt % aqueous ammonia solution. Excess solvents, impurities and side products such as shortmers can be removed from the crude product stream using chromatography. Ion exchange or reverse phase chromatography may also be used, in addition or in the alternative. The crude oligonucleotide solution is passed through a bed or column packed with a stationary phase to isolate the purified target. In the case of reverse phase chromatography, purification is conducted with the acid-labile protecting group, such as 5′-DMT, attached to the oligonucleotide.

d) Deprotection or Detritylation—Treatment with Acids

This step is undertaken to remove the acid-labile protecting groups (e.g., DMT). The removal of an acid-labile protecting group is called deprotection, and the removal of a trityl group is called detritylation. This step may be performed by contacting the oligonucleotide with an acid solution (e.g. 1-4% or 2-4% Trifluoroacetic acid (TFA) or Trichloroacetic acid (TCA)). Glacial acetic acid (AcOH) is also used for removing terminal acid-labile protecting groups from the oligonucleotide, but typically requires longer reaction times. The long reaction times under acidic conditions yield the possibility that side reactions will occur, such as depurination. Thus, AcOH is a less favored reagent for deprotection. The mechanism of cleaving DMT groups using TCA is shown in Equation 2, in which the nucleoside base and the remainder of the oligonucleotide chain are not depicted. It is known to conduct this deprotection in solution (as described in U.S. Pat. No. 10,618,931, for example), or via passage through a catalyst column or bed (as described in U.S. Pat. No. 5,808,042A, for example), or in an adsorptive, on-column manner (as described in U.S. Appln. Publn. No. 20220410035, for example).

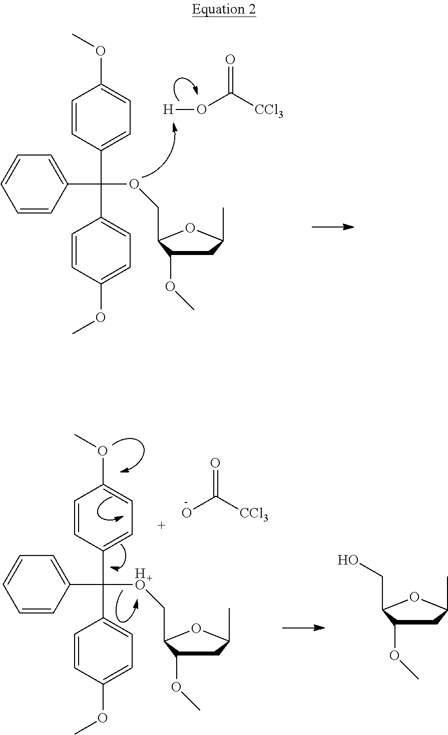

### f) Concentration and Formulation

The deprotected oligonucleotide can then be recovered; purified if necessary, using one or more of chromatography, precipitation, solvent evaporation, vacuum drying or other methods; and optionally further reacted or formulated into desired products.

Notably, steps (a) through (f) may be performed in any order that is logical, regardless of the order of their alphabetical labels. For example, it is possible to purify the feed solution (step d) at least partially before deprotecting the 5′ hydroxyl group (step c).

Several challenges are associated with oligonucleotide deprotection, however. For example, when using an acid for deprotection:
Some of the acids that may be used (e.g. TFA, TCA) are hazardous and require special care and handling during the operation and disposal.
In many cases, long contact times of the acid with the oligonucleotide are required to accomplish the deprotection or detritylation. At commercial scale, the reaction time may exceed several hours.
The long exposure of the acid to the oligonucleotide may cause side reactions which degrade the oligonucleotide, such as depurination.

Attempts to address these challenges are described in U.S. Pat. No. 10,618,931, for example, which describes cooling the oligonucleotide solution prior to adding an acid to avoid depurination. Lower reaction temperatures may result in longer reaction times, however.

Other examples of methods used to address these challenges are described in U.S. Appln. Publn. No. 20220410035, for example, which describes the use of anion exchange resin chromatography for on-column detritylation of oligonucleotides. That is, the protected oligonucleotide is adsorbed to the column followed by a detritylation step using an acid. An additional purification step is required to remove or neutralize the ammonia solution present in the crude oligonucleotide which is produced during the oligonucleotide cleavage from the solid synthesis support.

Yet another example is described in U.S. Pat. No. 5,808,042, which describes deprotection of oligonucleotides by contacting a pre-purified oligonucleotide solution with an ion exchange resin. This provides a faster reaction time and a flow-through process. Nevertheless, further treatment is required to remove ammonia, and the contact time between the resin and the oligonucleotide is relatively long.

It is apparent that a need remains for a deprotection process for organic molecules and in particular for a reliable oligonucleotide deprotection and purification process that are fast, simple, and suitable for research and industrial use.

### SUMMARY OF THE INVENTION

Accordingly, provided herein is a purification and deprotection process for crude acid-labile protected organic molecules, including oligonucleotides. Although the terms “DMT” and “trityl” are mentioned herein as acid-labile protecting groups, the process described herein is not limited to DMT or trityl-group protected molecules. Rather, the processes described herein are believed to be useful to remove any acid-labile protecting group, for example from the terminal 5′-OH of an oligonucleotide, as is set forth in greater detail below. In addition, the processes described herein address several specific problems associated with the purification and deprotection of oligonucleotides:
The crude oligonucleotide solution contains a high concentration of ammonia or another free base and protected oligonucleotides. The process described herein does not require any additional steps to remove basic impurities or salts that may be present in the crude oligonucleotide solution, as these are removed by the acidic resin.
Deprotection of the 5′ hydroxy group in the oligonucleotide, e.g., removal of a DMT group, does not require use of any acid such as TFA, TCA or glacial acetic acid. Thus, the use of these acids can be eliminated, resulting in faster reaction times, higher productivity, and the elimination of the production of halogenated and hazardous acid waste.
The process described herein is preferably carried out in a flow-through column or other bed format, rather than a batch mixture of resin beads in liquid. The contact time required to remove the acid-labile protecting group while flowing through a column is significantly reduced versus batch processing.

More specifically, the process described herein comprises the step of contacting a crude stream of protected organic molecules with a crosslinked acidic cation exchange resin catalyst in a batchwise process or in a continuous process, e.g., by the flowing the crude stream through a bed or column. The acid-labile group is removed from the protected organic molecules by a deprotection reaction that is catalyzed by the acidic catalyst resin. In the case of protected oligonucleotides, the 5′ acid-labile group, e.g., DMT, is removed by a deprotection reaction that is catalyzed by the acidic catalyst resin. When deprotecting oligonucleotides, the contact time of the crude stream with the catalyst in a continuous process is defined by a flow rate that is preferably in the range of 12 BV/hr to 240 BV/hr, more preferably 60 to 200 BV/hr. When deprotecting other organic molecules in a continuous process, the contact time to accomplish an acceptable level of deprotection varies with several factors, including the steric properties of the protected organic molecule, the hydrophobicity or hydrophilicity of the protected organic molecule, the identity of the protecting group, and the difference between the pKa of the acid catalyst resin and the pKa of the reagent that reacts to form the protecting group. In general, however, the contact time of the crude stream with the catalyst in a continuous process to deprotect other organic molecules is defined by a flow rate that is preferably in the range of 0.10 BV/hr to 240 BV/hr, more preferably 1.0 to 200 BV/hr or 60 to 200 BV/hr. As used herein, the term “BV” is an abbreviation for “bed volume.”

In one preferred process, the catalyst resin is in the form of a bed or column of crosslinked, oleum sulfonated, —SO₃H functionalized strongly acidic cation catalyst beads. The terms “bed” and “column” are synonymous and used interchangeably herein to describe the shape of a container for a wet or dry portion of resin beads, unless specifically stated otherwise in limited circumstances. Similarly, the terms “bed volume” (“BV”) and “column volume” (“CV”) are synonymous and used interchangeably herein to describe the volume of a wet or dry portion of resin beads, including the interstitial space between the beads, unless specifically stated otherwise in limited circumstances. In general, columns are cylindrical, and beds are not necessarily cylindrical.

Alternatively, another preferred process comprises the step of contacting the crude stream with a bed or column of crosslinked, —COOH functionalized weakly acidic catalysts to deprotect the organic molecules using a contact time defined by a flow rate that is preferably in the range of 12 BV/hr to 240 BV/hr for protected oligonucleotides such as DMT-protected oligonucleotides, or preferably longer contact times for other protected organic molecules.

When the deprotection is conducted in a batch process, procedures similar to those described in U.S. Pat. No. 5,808,042, e.g., may be used.

### BRIEF DESCRIPTION OF THE DRAWINGS

FIG. 1 is a schematic diagram of oligonucleotide synthesis using phosphoramidite chemistry, according to the prior art.

FIG. 2 is a flow chart comparing the process described herein to a process that is known in the art.

FIG. 3A is a high-pressure liquid chromatography (“HPLC”) trace of the crude tritylated oligonucleotide feed solution L1.

FIG. 3B is an expanded view of the HPLC trace of the crude oligonucleotide feed solution L1 shown in FIG. 3A.

FIG. 4A is an HPLC trace of the crude oligonucleotide feed solution L2 after passing through a bed of strong acid catalysts.

FIG. 4B is an expanded view of the HPLC trace of FIG. 4A, depicting the results for the crude oligonucleotide feed solution L2 after passing through a bed of strong acid catalysts.

FIG. 5A is an HPLC trace of the detritylated oligonucleotide solution L3 after the first passing through a bed of weak acid catalysts.

FIG. 5B is an expanded view of the HPLC trace of FIG. 5A, depicting the results for the detritylated oligonucleotide solution L3 after the first passing through a bead of weak acid catalysts.

FIG. 6A is an HPLC trace of the detritylated oligonucleotide solution L4 after the second passing through a bed of weak acid catalysts.

FIG. 6B is an expanded view of the HPLC trace of FIG. 6A, depicting the results for the detritylated oligonucleotide solution L4 after the second passing through a bed of weak acid catalysts.

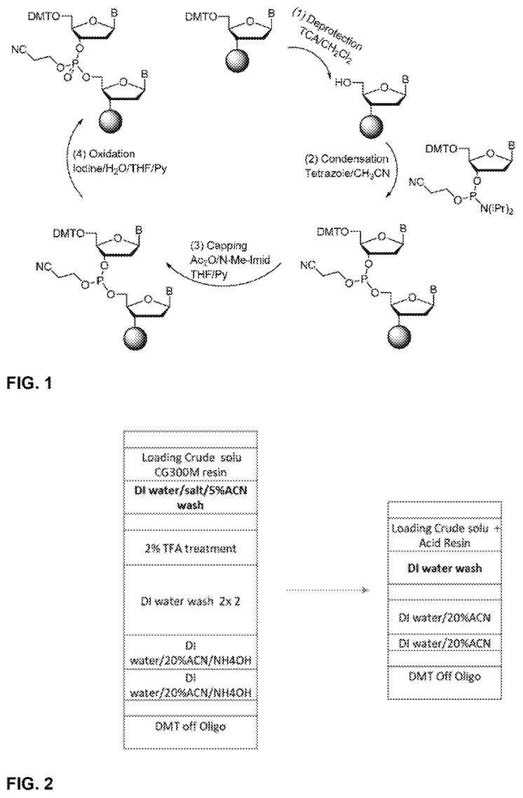

### DETAILED DESCRIPTION OF THE INVENTION

A method of deprotecting organic molecules, for example crude oligonucleotides, is provided herein. The method comprises contacting a crude acid-labile protected molecule (for example a 5′-DMT oligonucleotide mixture) with a crosslinked, acid-functionalized cation exchange resin catalyst.

Organic molecules that are suitable for use in the methods described herein include soluble organic molecules with a functional group that is capable of being protected by an acid-labile protecting group. Suitable organic molecules are soluble in water or any organic solvent. Among organic solvents, protic solvents and non-protic polar solvents are preferred. Water, alcohols, ethers, esters, and combinations of two or more of water, alcohols, ethers, and esters are more preferred solvents. Still more preferably, the organic solvents are low molecular weight solvents such as alcohols, ethers, and esters containing one or more branched or unbranched alkyl groups comprising from 1 to 4 carbon atoms. The products of the deprotection reaction are preferably also soluble in the same solvent(s) as the crude mixture of protected organic molecules.

Suitable organic molecules include one or more functional groups, such as for example hydroxyl, amino and thiol groups, that may be reacted with an acid-labile protecting group. Preferred organic molecules include specialty organic materials, for example pharmaceuticals; fragrances and flavors; dyes and pigments; agrichemicals such as pesticides, herbicides, and insecticides; specialty monomers; functional materials; and surfactants. More preferred organic molecules include biologically active molecules such as peptides and polynucleotides. Oligonucleotides, particularly 5′-protected oligonucleotides, are preferred water-soluble organic molecules.

In a preferred method, a mixture containing organic molecules that are protected with acid-labile groups is contacted with a crosslinked, oleum sulfonated —SO₃H functionalized strongly acidic cation catalyst. It has unexpectedly been found that crosslinked, oleum sulfonated —SO₃H functionalized strongly acidic cation catalysts deprotect the organic molecules more rapidly than other known methods.

In another preferred method, a crude oligonucleotide mixture is contacted with a crosslinked, oleum sulfonated —SO₃H functionalized strongly acidic cation catalyst. It has unexpectedly been found that crosslinked, oleum sulfonated —SO₃H functionalized strongly acidic cation catalysts deprotect 5′-protected oligonucleotides more rapidly than other known methods. The method is particularly effective to detritylate 5′-DMT protected oligonucleotides.

Crude, 5′-protected oligonucleotide solutions, as described above, may also include alkyl amides, ammonia, salts, organic solvents, shortmers, and other impurities. Accordingly, these are basic solutions having a pH greater than 7. The pH of the crude oligonucleotide solution may be greater than 9 and up to 12, 13, or 14.

A variety of acid-labile protecting groups are commonly used to prevent reaction at the functional groups of organic molecules, such as the 5′-OH group of an oligonucleotide. The methods described herein are suitable to remove acid-labile groups including, but not limited to, those described in U.S. Pat. No. 10,618,931. As used herein the term “protecting group” refers to a labile chemical moiety which is known in the art to protect reactive groups, including without limitation hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups are described generally in Greene's Protective Groups in Organic Synthesis, 4th edition, John Wiley & Sons, New York, 2007 (hereinafter “Greene”).

Suitable classes of protecting groups include, without limitation, ethers, esters, carbonates, benzyl-type groups, and other acid labile protecting groups that are known in the art. Specific examples of acid-labile hydroxyl protecting groups that are suitable for use in the processes described herein include, without limitation, carbonates, such as t-butyl carbonate and allyl carbonate; acetyl; t-butyl; t-butoxymethyl; methoxymethyl; tetrahydropyranyl; 1-ethoxyethyl; 1-(2-chloroethoxy)ethyl; p-chlorophenyl; 2,4-dinitrophenyl; benzyl; 2,6-dichlorobenzyl; diphenylmethyl; p-nitrobenzyl; bis(2-acetoxyethoxy)methyl (ACE); 2-trimethylsilylethyl; trimethylsilyl; triethylsilyl; t-butyldimethylsilyl; t-butyldiphenylsilyl; triphenylsilyl; [(triisopropylsilyl)oxy]methyl (TOM); benzoylformate; chloroacetyl; trichloroacetyl; trifluoroacetyl; pivaloyl; benzoyl; p-phenylbenzoyl; 9-fluorenylmethyl carbonate; mesylate; tosylate; triphenylmethyl (trityl); monomethoxytrityl (MMT); dimethoxytrityl (DMT); trimethoxytrityl; 1 (2-fluorophenyl)-4-methoxypiperidin-4-yl (FPMP); 9-phenylxanthine-9-yl (Pixyl); and 9-(p-methoxyphenyl)xanthine-9-yl (MOX). More commonly used hydroxyl protecting groups include, without limitation, benzyl; 2,6-dichlorobenzyl; t-butyldimethylsilyl; t-butyldiphenylsilyl; benzoyl; mesylate; tosylate; dimethoxytrityl (DMT); 9-phenylxanthine-9-yl (Pixyl); 9-(p-methoxyphenyl)xanthine-9-yl (MOX); and combinations of two or more of these protecting groups. An organic molecule comprising two or more functional groups may be protected by one or more acid-labile protecting groups that are the same or different.

The methods described herein are particularly well-suited to the removal of trityl groups (“detritylation”) of 5′-tritylated oligonucleotides. The term “trityl group” as used herein refers to triphenylmethyl (trityl); monomethoxytrityl; dimethoxytrityl (DMT); and trimethoxytrityl groups; and to combinations of two or more of these groups. Tritylation and detritylation reactions are known in the art. See, for example, Gait, cited above, and Greene, cited above.

In preparation for the deprotection reaction, the strongly or weakly acidic cation catalyst must be in protonated form. To ensure that a catalyst is protonated, it is treated by soaking in aqueous solution of a strong acid, for example hydrochloric acid, to remove any impurities and protonate any unprotonated acid sites on the catalyst, followed with soaking in one or more portions of deionized (DI) water to remove remaining impurities and residual acid. The aqueous acid solution is preferably dilute, for example, from about 0.001 to about 0.1M, from about 0.005M or about 0.01M to about 0.05M, or about 0.02M for a monoprotic acid, or equivalent pH for multiprotic acids. The time of exposure of the resin to the acid solution is preferably more than 1 h, more than 2 h, more than 4 h, more than 6 h, more than 8 h, or more than 12 h, to ensure that the resin is in H-form. The volume of acid solution preferably provides an excess of H+ cations, and the volume of DI water is preferably at least 50 times the volume of the catalyst beads. The impurities and residual acid are adequately removed when the pH of the last portion of DI water in which the catalyst was soaked is greater than 5.

Suitable catalysts for use in the processes described herein include sulfonated, crosslinked strongly acidic cation resins having one or more of the following properties:
(a) 10 to 85 weight percent copolymerized crosslinker units, based on dry weight of ion exchange resin, wherein monomers having two or more vinyl groups are preferred crosslinker units;
(b) 15 to 90 weight percent of one or more copolymerized monovinyl comonomer units, for example styrene units, based on the dry weight of the ion exchange resin, wherein the sum of the weight percentages of the copolymerized units of crosslinker and of monovinyl comonomer is 100 weight percent;
(c) no less than 5.0, preferably 5.0 to 7.0 milliequivalents sulfonic acid groups per gram, based on the dry weight of ion exchange resin;
(d) particle diameters of up to 1000 micrometers (μm), preferably 25 to 1000 μm, more preferably 50 to 1000 μm, more preferably 100 to 1000 μm, even more preferably 200 to 1000 μm, still more preferably 300 to 1000 μm.

The acid-labile protected organic molecules, for example the 5′-protected-oligonucleotides, are deprotected by exposing them to the catalyst resin in a batch process, or in a continuous process by flowing them over the catalyst, for example by passing a solution of protected molecules through a bed or column packed with the acidic cation catalyst prepared as described above. An additional portion of the solvent of the crude protected mixture, or a portion of a secondary solvent, or a mixture of the solvent with the secondary solvent, may also be added to the bed or column to further elute the solution of deprotected molecules. Water is a preferred secondary solvent. Aqueous solutions, for example salt solutions or buffer solutions, are not preferred, as ions in these solutions may exchange with those in the resin. Other preferred secondary solvents include alkanols, such as methanol and ethanol. Non-protic solvents such as acetonitrile may be more preferable in certain cases. The amount of solvent, secondary solvent, or solvent mixture for further elution is sufficient to rinse the feed liquid from the column, for example 1 to 2 column volumes. In a batch or continuous process, the catalyst may be separated from the reaction mixture to provide the solution of deprotected organic molecules by any suitable means, for example by decanting, sieving, or filtration; by rinsing the catalyst with water or a basic solution such as an ammonia solution; by blowing air through the catalyst bed, including, e.g., cannulation; or by replacing the solution of deprotected organic molecules with additional crude stream.

As described above, the contact time of a protected organic molecule with a strongly acidic catalyst resin to achieve an acceptable level of deprotection varies with several factors. Nevertheless, the contact time of a protected oligonucleotide solution with the strongly acidic catalyst resin in order to achieve essentially complete deprotection may be defined by a flow rate range of 12 BV/hr to 240 BV/hr. To improve efficiency, the flow rate range is preferably no less than 12 BV/hr, more preferably no less than 20 BV/hr, even more preferably from 60 BV/hr to 200 BV/hr. When the deprotection of the organic molecule is conducted as a batch process, the amount of acid sites in the catalyst resin is preferably greater than 1 mol %, from 1 to 10 mol %, or up to and including 100 mol %, based on the number of moles of protected functional groups in the quantity of molecules to be deprotected. When ammonia or other basic impurities (if any) must be removed from a crude oligonucleotide solution or from a reaction mixture containing another organic molecule, an amount of acid sites that is greater than or equal to the stoichiometric equivalent of the amount of ammonia and other basic impurities is required, in addition to the amount of acid sites that are required for the desired level of catalytic activity. For example, the number of acid sites in the catalyst resin is preferably greater than or equal to the number of equivalents of ammonia and other basic impurities plus 1 to 10% of the number of moles of protecting groups to be removed from the oligonucleotide or other organic molecule. The contact time of the oligonucleotide feed solution with the catalyst is preferably less than 5 minutes, more preferably less than 4, 3, 2, or 1 minute. The contact time of a feed solution containing a different protected organic molecule may be longer, as described above with respect to the factors affecting suitable flow rates. The completeness of the deprotection reaction in the eluted oligonucleotide solution may be assessed via a number of analytical methods, including, without limitation, high-pressure liquid chromatography (HPLC), UV-vis spectroscopy, and thin-layer or paper chromatography. Those of skill in the art are also capable of using these analytical methods to determine a suitable contact time or to assess the completeness of the deprotection of other organic molecules.

Thus, in addition to deprotecting the oligonucleotides, the step of exposing the crude protected oligonucleotide solution to the strongly acidic cation exchange resin catalyst also purifies the crude solution by removing ammonia, other basic impurities, and salts via stoichiometric reaction with or without adsorption onto the catalyst.

A crude stream flowed through a bed or column of strongly acidic cation exchange resin catalyst, or one that is exposed to the catalyst in a batch process, generates a product stream comprising the deprotected organic molecule, for example the oligonucleotide. The product stream comprising the deprotected and purified organic molecules may be used as-is; alternatively, it may be concentrated, processed or reacted further to produce an end product.

It has now surprisingly been discovered that a cross-linked, strongly acidic, preferably —SO₃H functionalized cation exchange resin catalyst can be used for purification and deprotection of crude oligonucleotides or other organic molecules in a single step. Significantly, with respect to oligonucleotides, the traditional process requires two different steps for deprotection and purification. These are: 1) removal of ammonia, basic impurities, and salts from the crude mixture by evaporation or washing with solvent or buffers; and 2) cleavage of acid-labile protecting groups from terminal 5′ position of the oligonucleotide by reacting with 1 to 4% trifluoroacetic acid or trichloroacetic acid, by reacting with 80% glacial acetic acid, or by adsorbing the 5′-protected oligonucleotide onto an anion exchange resin followed by introduction of aqueous acid to remove acid labile groups and subsequently by desorption of the deprotected oligonucleotide from the resin. In the process described herein, however, a single-step purification and deprotection is achieved by using a strongly acidic cation exchange resin catalyst. Those of skill in the art equipped with the teachings provided herein can readily apply these principles to achieve the deprotection and purification of other organic molecules.

Further provided herein is a method comprising contacting a crude solution containing an acid-labile protected organic molecule, for example a 5′-protected oligonucleotide, with a crosslinked, —COOH functionalized weakly acidic cation exchange resin catalyst. It has now unexpectedly been found that crosslinked, —COOH functionalized weakly acidic cation exchange resin catalysts also deprotect organic molecules such as 5′-protected oligonucleotides, for example detritylating 5′-DMT oligonucleotides, more efficiently than prior art methods.

As described above with respect to strongly acidic catalysts, to ensure that the weakly acidic cation exchange resin catalyst is protonated, it may first be soaked in aqueous hydrochloric acid to remove any impurities and to activate any unactivated acid sites on the catalyst, followed with soaking in DI water to remove impurities and residual acid. Suitable acid solutions, volumes and concentrations of acid solution, volumes of DI water, flow rates, and soaking times are as described above with respect to strongly acidic cation exchange resin catalysts. Suitable catalysts for use in this method include weakly acidic cationic exchange resins having one or more of the following properties:
(a) 4 to 100 weight percent copolymerized crosslinker units, based on dry weight of ion exchange resin, wherein the remainder copolymerized residues are one or more monovinyl comonomers, for example methacrylic acid, such that the sum of the weight percentages of the copolymerized units of crosslinker and of monovinyl comonomer is 100 weight percent; and wherein the suitable and preferred crosslinker units are as described above with respect to the strongly acidic cation exchange resin; and wherein the suitable and preferred monovinyl comonomers are selected from the group consisting of acrylic acid; methacrylic acid; alkyl acrylates, preferably methyl acrylate or ethyl acrylate; alkyl methacrylates, preferably methyl methacrylate or ethyl methacrylate; and acrylonitrile;
(b) no less than 4.0, preferably 4.0 to 12.0 milliequivalents acetic acid groups per gram, based on dry weight of ion exchange resin; and preferably 5.0 to 12.0 milliequivalents, more preferably 7.0 to 12.0 milliequivalents, more preferably 9.0 to 12.0 milliequivalents, even more preferably 10.0 to 12 milliequivalents; and
(c) particle diameters of up to 1000 μm, preferably 25 to 1000 μm, more preferably 50 to 1000 μm, more preferably 100 200 to 1000 μm, even more preferably 200 to 1000 μm, still more preferably 300 to 1000 μm.

The step of contacting the stream of protected organic molecules with the strongly or weakly acidic cation exchange resin catalyst not only purifies the crude solution of organic molecules by removing any ammonia, other basic impurities, and salts that may be present, but in addition the acid-labile protected molecules such as 5′-protected oligonucleotides are also deprotected by passing them through a bed or column packed with the resin prepared as described above by treatment with HCl solution followed by washing with an appropriate solvent or solvent mixture. For example, DI water may then be added to the column to elute the deprotected oligonucleotides from the column. As described above, the contact time of the crude stream with the catalyst in a continuous process to deprotect organic molecules is defined by a flow rate that is preferably in the range of 0.10 BV/hr to 240 BV/hr, more preferably 1.0 to 200 BV/hr or 60 to 200 BV/hr. The contact time for the purification and deprotection of oligonucleotides may be defined by a flow rate range of 12 BV/hr to 240 BV/hr. To improve the efficiency of the oligonucleotide deprotection and purification, the flow rate range is preferably no less than 12 BV/hr, more preferably no less than 20 BV/hr, even more preferably 60 BV/hr to 200 BV/hr. The means of and conditions for obtaining the deprotected organic molecules after reaction with the weakly acidic cation exchange resin catalyst are as described above with respect to the strongly acidic cation exchange resin catalyst.

Accordingly, it has now further been discovered that-COOH functionalized catalysts can also be used for purification and deprotection of organic molecules such as crude oligonucleotides in one step. The traditional process, as described above with respect to the process employing strongly acidic cation exchange resin catalysts, requires two separate steps to deprotect and purify oligonucleotides. In the process described herein, however, a single-step purification-detritylation is achieved by using a weakly acidic cation exchange resin catalyst.

More specifically, when ammonia or other basic impurities (if any) must be removed from a crude oligonucleotide solution or from a reaction mixture containing another protected organic molecule, an amount of acid sites that is greater than or equal to the stoichiometric equivalent of the amount of ammonia and other basic impurities is required, in addition to the amount of acid sites that are required for the desired level of catalytic activity. For example, the number of acid sites in the catalyst resin is preferably greater than or equal to the number of equivalents of ammonia and other basic impurities plus 1 to 10% of the number of moles of protecting groups to be removed from the oligonucleotide or other organic molecule. The contact time of the oligonucleotide feed solution with the catalyst is preferably less than 5 minutes, more preferably less than 4, 3, 2, or 1 minute, as described above with respect to the strongly acidic catalysts. It is to be expected, however, that the optimal contact times of the crude solutions with a weakly acidic catalyst may be longer, for example up to 10 minutes or even longer, in the case of other protected organic molecules. The completeness of the deprotection reaction in the eluted oligonucleotide solution may be assessed via a number of analytical methods, including, without limitation, high-pressure liquid chromatography (HPLC), UV-vis spectroscopy, and thin-layer or paper chromatography. Those of skill in the art are also capable of using these analytical methods to determine a suitable contact time or to assess the completeness of the deprotection of other organic molecules.

Thus, in addition to deprotecting the organic molecules, the step of exposing the crude protected oligonucleotide solution to the weakly acidic cation exchange resin catalyst also purifies the crude solution by removing ammonia, other basic impurities, and salts via stoichiometric reaction with or without adsorption onto the catalyst.

The process described herein provides a significant impact on purification of crude, protected oligonucleotides by one or more of removing extra purifying or washing steps; removing the use of hazardous TFA/TCA; and removing extra washing with DI water/buffer for complete removal of traces of acid before the deprotected oligonucleotide is eluted. Although the deprotection reaction is a catalytic one, the resin beads are expected to be single use only; thus, there is no need to remove any absorbed ammonia or regenerate the beads. Alternatively, the resin beads may be regenerated. The improvements that result from the processes described herein are illustrated schematically in FIG. 2, wherein the abbreviation “ACN” stands for “acetonitrile.”

In the processes described herein, the temperature of the feed stream of the crude oligonucleotide is preferably from 5 to 50° C., more preferably from 5 to 40° C., even more preferably from 10 to 30° C. Alternatively, the processes may preferably be conducted at a temperature from 5° C. to room temperature or from 5° C. to 27° C. This range of temperatures enables a balance between the deprotection reaction efficiency and the reduction of side reactions such as depurination of oligonucleotides. The temperature of the feed stream of other organic molecules, which may be dissolved in an organic solvent or solvent mixture, is from −78° C. to 120° C., from 0 to 100° C., from 5 to 50° C., from 5 to 40° C., or from 10 to 30° C.

Any strongly acidic cation exchange resin catalyst may be suitable for use in the processes described herein. Non-limiting examples of suitable catalysts are described in Kunin, Robert. Ion Exchange Resins. United States: R. E. Krieger, 1990 (hereinafter Kunin). Preferred strongly acidic cation exchange resins comprise a cross-linked polymer matrix with functional groups. The polymer matrix is cross-linked by one or more crosslinking comonomers. Suitable crosslinking comonomers are also described in Kunin. Preferred crosslinking comonomers are selected from the group consisting of multifunctional vinyl monomers, such as multifunctional or multivinyl styrenic vinyl or (meth)acrylate monomers. Preferably, the strongly acidic cation exchange resin catalyst includes only one crosslinking comonomer. More preferably, the sole crosslinking comonomer is divinyl benzene.

Suitable monovinyl comonomers are also described in Kunin. Preferred monovinyl comonomers include, without limitation, styrenics, acrylates, methacrylates and acrylonitriles. Preferably, the monovinyl comonomers comprise styrene. More preferably, the strongly acidic cation exchange resin catalyst includes only one monovinyl comonomer. Still more preferably, the sole monovinyl comonomer is styrene.

Preferred strongly acidic cation exchange resin catalysts comprise 10 to 85 weight percent of copolymerized crosslinking comonomer(s), more preferably 10 to 50 weight percent, still more preferably 10 to 25 weight percent. The amount of copolymerized monovinyl comonomer(s) is complementary to the amount of copolymerized crosslinking comonomer(s), that is, the sum of the weight percentages of the copolymerized comonomers is 100 weight percent. The weight percentages are based on the total weight of the dry acidic cation exchange resin.

The strongly acidic cation exchange resin catalyst particles have particle diameters of up to 1000 micrometers (μm), preferably 25 to 1000 μm, more preferably 50 to 1000 μm, more preferably 100 to 1000 μm, still more preferably 200 to 1000 μm, still more preferably 300 to 1000 μm.

Comonomers of monovinyl comonomers and crosslinking comonomer(s) may be sulfonated according to known methods to provide a strongly acidic cation exchange resin. See Kunin, for example. Suitable strongly acidic cation exchange resins for use herein include no less than 5.0, preferably 5.0 to 7.0 milliequivalents (meq) of sulfonic acid groups per gram, based on the dry weight of the strongly acidic cation exchange resin, more preferably 5.0 to 6.0 meq/g, still more preferably 5.0 to 5.5 meq/g.

Similarly, any weakly acidic cation exchange resin may be suitable for use in the processes described herein. Non-limiting examples of suitable weakly acidic cation exchange resins are described in Kunin. Preferred weakly acidic cation exchange resins are copolymers of one or more monovinyl comonomers and one or more crosslinking comonomers. Suitable and preferred monovinyl and crosslinking comonomers are as described above with respect to strongly acidic cation exchange resins. Preferably, the weakly acidic cation exchange resin includes only one crosslinking comonomer. More preferably, the sole crosslinking comonomer is divinyl benzene. Also preferably, the monovinyl comonomers comprise (meth)acrylates. More preferably, the weakly acidic cation exchange resin includes only one monovinyl comonomer. Still more preferably, the sole monovinyl comonomer is acrylic acid.

Preferred weakly acidic cation exchange resins comprise 4 to 100 weight percent of copolymerized crosslinking comonomer(s), more preferably 4 to 50 weight percent, still more preferably 4 to 25 weight percent. The amount of copolymerized monovinyl comonomer(s) is complementary to the amount of copolymerized crosslinking comonomer(s), that is, the sum of the weight percentages of the copolymerized comonomers is 100 weight percent. The weight percentages are based on the total weight of the dry weakly acidic cation exchange resin.

Comonomers of monovinyl comonomer(s) and crosslinking comonomer(s) may be carboxylated according to known methods to provide a weakly acidic cation exchange resin. See Kunin, for example. Suitable weakly acidic cation exchange resins for use herein include no less than 4.0, or 4.0 to 12.0 milliequivalents acetic acid groups per gram, based on dry weight of ion exchange resin; and preferably 5.0 to 12.0 milliequivalents, more preferably 7.0 to 12.0 milliequivalents, still more preferably 9.0 to 12.0 milliequivalents, even more preferably 10.0 to 12 milliequivalents.

The suitable and preferred particle sizes of the weakly acidic cation exchange resins are as set forth above with respect to the strongly acidic cation exchange resin particle sizes.

Suitable amounts of strongly and weakly acidic cation exchange resin catalysts for deprotecting the organic molecules may be determined for a known concentration of organic molecules and a desired exposure time based on well-known principles of kinetics for acid-catalyzed reactions. When it is desired to deprotect the organic molecules catalytically and to remove basic impurities in a single step, suitable amounts of acid-functionalized resin may be determined stoichiometrically. Briefly, the number of acid equivalents in the resin should be greater than or equal to the number of equivalents of basic impurities in the crude stream of protected organic molecules.

Preferably, the amount of acid-labile protecting group that is removed is greater than 50 mol %, greater than 75 mol %, greater than 90 mol %, up to 100 mol %, or 100 mol %, based on the total number of protecting groups in the portion of protected organic molecules that is contacted with the strongly or weakly acidic cation exchange resin catalyst.

Suitable eluants for removing the deprotected organic molecules from the strongly or weakly acidic cation exchange resin catalysts (“rinse eluants”) include, without limitation, the solvent(s) in which the protected organic molecules were contacted with the catalyst, DI water, and, less preferably, buffer solutions. The preferred eluant for deprotected oligonucleotides is DI water. The amount of eluant is determined by detecting the deprotected organic molecules in subsequent aliquots of eluted solution, until the cumulative amount of organic molecules collected is acceptable, or until the concentration of organic molecules in the last aliquot of eluted solution is acceptably low.

The rinse eluant is preferably fed to the catalyst bed in a manner described above with respect to the feed solution. The contact time may be defined by a flow rate range of 12 BV/hr to 240 BV/hr.

The eluted solution of deprotected organic molecules may be subject to further processes that are well known in the art, such as for example further purification, concentration, formulation, or reactions.

Significantly, the methods described herein may be performed at any appropriate juncture in a series of synthesis and purification processes, provided in the case of oligonucleotide deprotection that the nucleotide bases are connected in the correct order to form the protected oligonucleotide, and provided that the protected oligonucleotide is cleaved from the solid-phase synthesis support. For example, it may be desirable to react the protected organic molecules further before deprotecting them. Alternatively, it may be desirable to remove excess ammonia and other impurities from the crude feed solution of protected oligonucleotides before performing a deprotection or other further reaction. The effectiveness of the methods described herein is independent of the order in which the process steps are performed.

The following examples are provided to describe the invention in further detail. These examples, which set forth specific embodiments and a preferred mode presently contemplated for carrying out the invention, are intended to illustrate and not to limit the invention.

### Examples

### Methods

### A. HPLC Analysis

HPLC analysis was carried out using ThermoScientific DNAPac™ analytical AIEX HPLC column available from Thermo Fisher Scientific, Inc., of Waltham, MA, using the following mobile phases:
Mobile Phase A: 75% aq. solution of 25 mM sodium phosphate buffer (pH 7.5) containing 25% acetonitrile.
Mobile Phase B: 75% aq. solution of 25 mM sodium phosphate buffer, pH 7.5+400 mM sodium perchlorate containing 25% acetonitrile.
Analysis of oligonucleotide solution for DMT-On (protected) and DMT-Off (deprotected) content was carried out using a gradient of Mobile phase B 13% to 100% over a period of 57 minutes. The DMT-On and DMT-Off peaks were easily separated using this gradient.

### B. Crude Oligonucleotide Feed Solution

A crude oligonucleotide feed solution was obtained via a synthesis procedure described in U.S. Pat. No. 10,618,931 after it was cleaved from solid support post-solid phase synthesis into an aqueous ammonia solution. Preferably, the concentration of the ammonia in the aqueous ammonia solution was from 28 to 30 wt % or from 30 to 33 wt %. The maximum range of the ammonia concentration was from 25 to 35 wt %, based on the total weight of the aqueous ammonia solution.

The crude oligonucleotide feed solution (150 μl) containing approximately 30 mg/ml of 5′-tritylated oligonucleotides was diluted with DI water to 2.5 ml. The UV absorbance of the diluted solution was 38.14 absorbance units at 260 nm. The pH of the diluted solution was approximately 10.58.

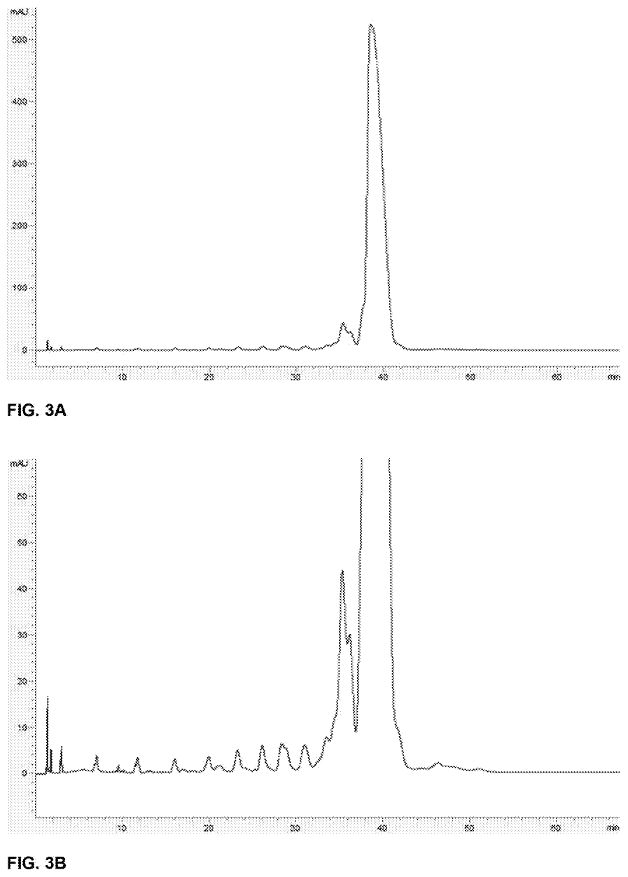

The diluted crude oligonucleotide feed solution was labeled as L1 and the HPLC analysis of solution L1 was carried out. This HPLC trace is shown in FIG. 3A, and an expanded view of the trace is shown in FIG. 3B.

### Experiments

A. One Step Purification-Detritylation with Strongly Acidic Cationic Exchange Resin and the Column

Sulfonated cross-linked strongly acidic cationic exchange resins were supplied by DuPont Water Solutions of Wilmington, DE (hereinafter “DuPont”). The resins had 16% percent copolymerized crosslinker units, based on dry weight of ion exchange resin; 5.0 to 7.0 milliequivalents sulfonic acid groups per gram, based on dry weight of ion exchange resin; and particle diameters of 300 to 1000 μm. The resin beads were soaked in 0.02M aqueous HCl overnight. The depleted acid solution was drained, and the acid-treated beads were then soaked overnight in sufficient DI water to create a slurry of beads with a significant amount of supernatant fluid. Total volume of the beads was 2.69 cm³, and 2.49 g was the weight of this wet cake. These beads were packed in a 6 ml syringe or “column” fitted with a porous frit at the bottom.

The diluted crude oligonucleotide feed solution was then transferred to the strongly acidic cationic exchange resin-packed column. The solution was allowed to flow through the column under gravity in such a way that the solution was eluted dropwise. The total time for the solution to be eluted from the column was <1 min. The flow rate was calculated to be 122 BV/hr.

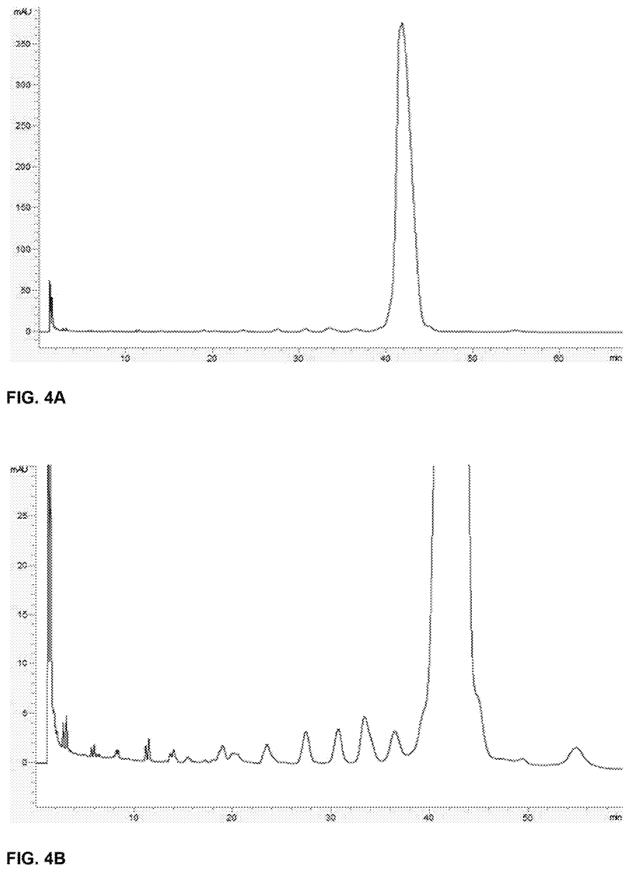

The solution after passing the strongly acidic cationic exchange resin packed column was labeled as L2. The UV absorbance of solution L2 was approximately 29.80 absorbance units at 260 nm. The pH of the eluted solution L2 was approximately 3.06. The HPLC analysis of solution L2 was carried out. This HPLC trace is shown in FIG. 4A, and an expanded view of the trace is shown in FIG. 4B.

B. One Step Purification-Detritylation with Weakly Acidic Cationic Exchange Resin and the Column

Weakly acidic cationic exchange resins were supplied by DuPont. The resins comprised copolymerized crosslinker; 7.0-12.0 milliequivalents acetic acid groups per gram, based on dry weight of ion exchange resin; and had particle diameters of 300 to 1000 μm. The resin beads were soaked in 0.02M aqueous HCl overnight. The depleted HCl solution was drained, and the acid-treated beads were then soaked in DI water overnight. The total volume of the beads was 2.48 cm³, and 2.39 g was the weight of this wet cake. These beads were packed in a 6 ml syringe fitted with a porous frit at the bottom.

The diluted crude oligonucleotide feed solution was then transferred to the weakly acidic cationic exchange resin-packed column. The solution was allowed to flow through the column under gravity in such a way that the solution was eluted dropwise. The total time for the solution to be eluted from the column was <1 min. The flow rate was calculated to be approximately 120 BV/hr.

The solution after passing the weakly acidic cationic exchange resin packed column was labeled as L3. The UV absorbance of solution L3 was approximately 24.50 absorbance units at 260 nm. The pH of the eluted solution L3 was approximately 5.80.

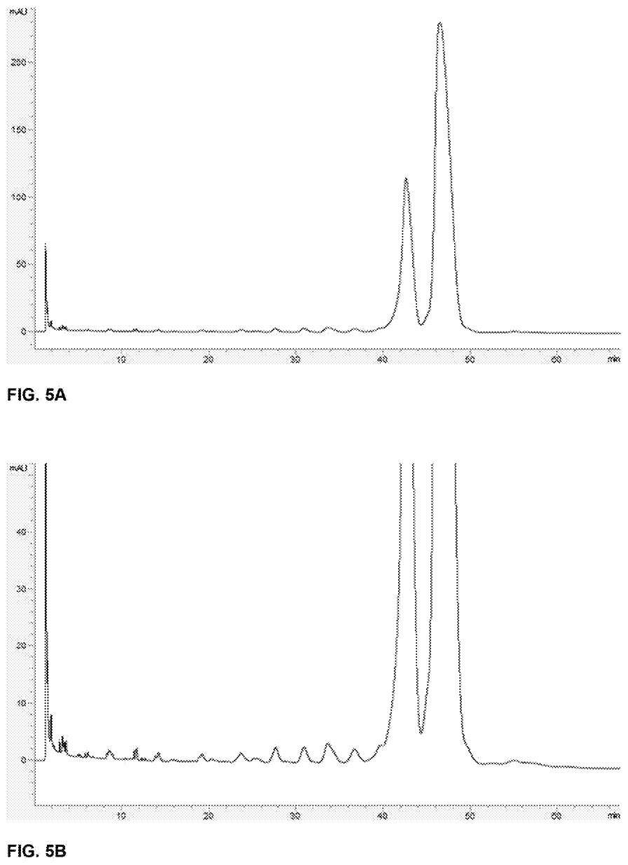

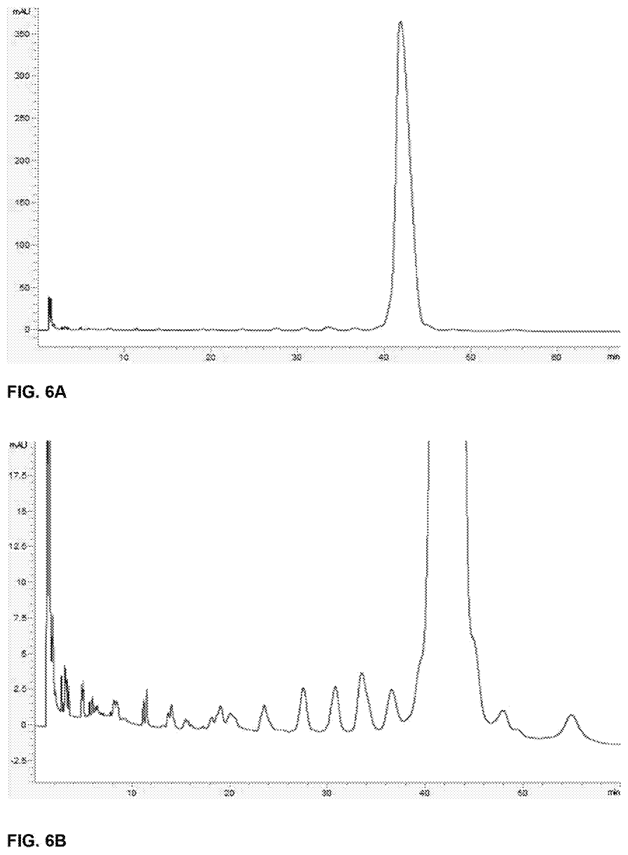

An aliquot (0.6 ml) of L3 solution was analyzed by HPLC. This HPLC trace is shown in FIG. 5A, and an expanded view of the trace is shown in FIG. 5B. The remaining L3 solution was loaded to the spent weakly acidic cationic exchange resin packed column without intervening regeneration or other treatment. It was allowed to elute one drop at a time under gravity. The solution after passage through the spent weakly acidic cationic exchange resin packed column was labeled as L4. The UV absorbance of solution L4 was approximately 28.50 absorbance units at 260 nm. The HPLC analysis of solution L4 was carried out. This HPLC trace is shown in FIG. 6A, and an expanded view of the trace is shown in FIG. 6B.

### Results

In FIGS. 3A and 3B, the HPLC trace of the crude oligonucleotide feed solution L1, which includes the DMT-On oligonucleotide, shows two distinct peaks. The first peak (approximately 35 minutes) is due to incomplete oligonucleotide sequences with DMT-Off and the second peak (approximately 38 minutes) is due to the oligonucleotides with DMT-On.

In FIGS. 4A and 4B, the HPLC trace of solution L2 only has one peak (approximately 42 minutes) due to the DMT-Off oligonucleotides. A peak due to DMT-On oligonucleotides was not detected, indicating a full conversion to oligonucleotides with DMT-off, i.e., deprotected or detritylated oligonucleotides. Only one pass through the column is required due to the presence of strongly acidic —SO₃H functional groups on the bead surface. Kinetics of deprotection of DMT groups is much faster than a corresponding —COOH functional resin due to strongly acidic nature of the —SO₃H groups.

In FIGS. 5A and 5B, the HPLC trace of solution L3 has two peaks. The first peak (approximately 42 minutes) is due to the DMT-Off oligonucleotides and the second peak (approximately 46 minutes) is the oligonucleotides with DMT-On. One pass through the weakly acidic cationic exchange resin packed column has not yet fully deprotected the DMT-On oligonucleotides.

In FIGS. 6A and 6B, the HPLC trace of solution L4 has one peak (approximately 42 minutes) due to the DMT-Off oligonucleotides. A peak due to DMT-On oligonucleotides was not detected, indicating a full conversion to DMT-Off oligonucleotides.

While certain of the preferred embodiments of this invention have been described and specifically exemplified above, it is not intended that the invention be limited to such embodiments. Various modifications may be made without departing from the scope and spirit of the invention, as set forth in the following claims.

1. A method of deprotecting an acid-labile group protected oligonucleotide comprising
(a) contacting a feed stream comprising the acid-labile group protected oligonucleotide with an acidic cation exchange catalyst to produce a product stream comprising a deprotected oligonucleotide, wherein said acidic cation exchange catalyst comprises:
i. 4 to 100 weight percent copolymerized crosslinker units, based on the dry weight of ion exchange catalyst; and
ii. no less than 4.0 milliequivalents of acid groups per gram, based on the dry weight of ion exchange catalyst; and
(b) separating the product stream from the acidic cation exchange catalyst.

2. The method of claim 1, wherein the acidic cation exchange catalyst is a strongly acidic cation resin catalyst.

3. The method of claim 2, wherein the strongly acidic cation resin catalyst has one or more of the following properties:
(a) 10 to 85 weight percent copolymerized crosslinker units, based on dry weight of ion exchange resin;
(b) 15 to 90 weight percent of one or more copolymerized monovinyl comonomer units, based on the dry weight of the ion exchange resin, wherein the sum of the weight percentages of the copolymerized units of crosslinker and of monovinyl comonomer is 100 weight percent;
(c) no less than 5.0 milliequivalents sulfonic acid groups per gram, based on the dry weight of ion exchange resin; and
(d) is in the form of beads having a particle diameter of up to 1000 micrometers (mm).

4. The method of claim 2, wherein the strongly acidic cation exchange resin catalyst comprises from 5 to 7 milliequivalents of acid groups per gram of dry ion exchange resin.

5. The method of claim 1, wherein the acidic cation exchange catalyst is a weakly acidic cation exchange resin catalyst.

6. The method of claim 5, wherein the weakly acidic cation exchange resin catalyst has one or more of the following properties:
(a) 4 to 100 weight percent copolymerized crosslinker units, based on dry weight of ion exchange resin, wherein the remainder copolymerized residues are one or more monovinyl comonomers, such that the sum of the weight percentages of the copolymerized units of crosslinker and of monovinyl comonomer is 100 weight percent; and wherein the crosslinker units are monomers having two or more vinyl groups; and wherein the monovinyl comonomers are selected from the group consisting of acrylic acid; methacrylic acid; alkyl acrylates; alkyl methacrylates; and acrylonitrile;
(b) no less than 4.0 milliequivalents acetic acid groups per gram, based on dry weight of ion exchange resin; and
(c) is in the form of beads having a particle diameter of up to 1000 mm.

7. The method of claim 5, wherein the weakly acidic cation exchange resin catalyst comprises no less than 7 milliequivalents per gram of acid groups.

8. The method of claim 3, wherein the beads of the acidic cation exchange catalyst are disposed in a bed, wherein the bed has a volume, and wherein the flow rate of the feed stream through the volume of the beads is 12 BV/hr to 240 BV/hr.

9. The method of claim 8, wherein the flow rate of the feed stream is 60 BV/hr to 200 BV/hr.

10. The method of claim 1, wherein said acid-labile group protected oligonucleotide is a 5′-protected oligonucleotide.

11. The method of claim 10, wherein said acid-labile group protected oligonucleotide is protected by an acid-labile protecting group selected from the group consisting of acetyl; t-butyl; t-butoxymethyl; methoxymethyl; tetrahydropyranyl; 1-ethoxyethyl; 1-(2-chloroethoxy)ethyl; p-chlorophenyl; 2,4-dinitrophenyl; benzyl; 2,6-dichlorobenzyl; diphenylmethyl; p-nitrobenzyl; bis(2-acetoxyethoxy)methyl (ACE); 2-trimethylsilylethyl; trimethylsilyl; triethylsilyl; t-butyldimethylsilyl; t-butyldiphenylsilyl; triphenylsilyl; [(triisopropylsilyl)oxy]methyl (TOM); benzoylformate; chloroacetyl; trichloroacetyl; trifluoroacetyl; pivaloyl; benzoyl; p-phenylbenzoyl; 9-fluorenylmethyl carbonate; mesylate; tosylate; triphenylmethyl (trityl); monomethoxytrityl (MMT); dimethoxytrityl (DMT); trimethoxytrityl; 1 (2-fluorophenyl)-4-methoxypiperidin-4-yl (FPMP); 9-phenylxanthine-9-yl (Pixyl); and 9-(p-methoxyphenyl)xanthine-9-yl (MOX) groups; and combinations of two or more of these groups.

12. The method of claim 11, wherein said acid-labile protecting group is selected from the group consisting of triphenylmethyl (trityl); monomethoxytrityl; dimethoxytrityl (DMT); and trimethoxytrityl groups; and combinations of two or more of these groups.

13. The method of claim 1, wherein said feed stream additionally comprises one or more of ammonium hydroxide, an alkyl amine, a salt, or a different free base; or wherein the pH of the product stream is less than 7; or wherein the amount of protecting group that is removed is greater than 50%; or wherein the temperature of the feed stream is from 5° C. to 50° C.; or wherein the acidic cation exchange catalyst resin is in the form of beads having particle diameters of 300 to 1000 μm.

14. (canceled)

15. (canceled)

16. (canceled)

17. The method of claim 1, wherein the product stream is separated from the catalyst by displacement with an eluant stream; and optionally wherein the eluant stream comprises one or more of water; the feed stream; a buffer solution; the product stream; and a non-reactive gas such as one or more of air, nitrogen, or argon.

18. (canceled)

19. (canceled)

20. The method of claim 6, wherein the beads of the acidic cation exchange catalyst are disposed in a bed, wherein the bed has a volume, and wherein the flow rate of the feed stream through the volume of the beads is 12 BV/hr to 240 BV/hr.

21. A method of deprotecting an acid-labile group protected organic molecule comprising:
(a) contacting a feed stream comprising the acid-labile group protected organic molecule with an acidic cation exchange catalyst to produce a product stream comprising a deprotected organic molecule, wherein said acidic cation exchange catalyst comprises:
i. 4 to 100 weight percent copolymerized crosslinker units, based on the dry weight of ion exchange catalyst; and
ii. no less than 4.0 milliequivalents of acid groups per gram, based on the dry weight of ion exchange catalyst; and
(b) separating the product stream from the acidic cation exchange catalyst.

22. The method of claim 21, wherein the acidic cation exchange catalyst is a strongly acidic cation resin catalyst.

23. The method of claim 22, wherein the strongly acidic cation resin catalyst has one or more of the following properties:
(a) 10 to 85 weight percent copolymerized crosslinker units, based on dry weight of ion exchange resin;
(b) 15 to 90 weight percent of one or more copolymerized monovinyl comonomer units, based on the dry weight of the ion exchange resin, wherein the sum of the weight percentages of the copolymerized units of crosslinker and of monovinyl comonomer is 100 weight percent;
(c) no less than 5.0 milliequivalents sulfonic acid groups per gram, based on the dry weight of ion exchange resin; and
(d) is in the form of beads having a particle diameter of up to 1000 micrometers (mm).

24. The method of claim 21, wherein the acidic cation exchange catalyst is a weakly acidic cation exchange resin catalyst.

25. The method of claim 24, wherein the weakly acidic cation exchange resin catalyst has one or more of the following properties:
(a) 4 to 100 weight percent copolymerized crosslinker units, based on dry weight of ion exchange resin, wherein the remainder copolymerized residues are one or more monovinyl comonomers, such that the sum of the weight percentages of the copolymerized units of crosslinker and of monovinyl comonomer is 100 weight percent; wherein the crosslinker units are monomers having two or more vinyl groups; and wherein the monovinyl comonomers are selected from the group consisting of acrylic acid; methacrylic acid; alkyl acrylates; alkyl methacrylates; and acrylonitrile;
(b) no less than 4.0 milliequivalents acetic acid groups per gram, based on dry weight of ion exchange resin; and
(c) is in the form of beads having a particle diameter of up to 1000 mm.

26. The method of claim 21, wherein said acid-labile group protected organic molecule comprises one or more protected functional groups selected from the group consisting of hydroxyl, amino and thiol groups.

27. The method of claim 26, wherein said acid-labile group protected organic molecule is selected from the group consisting of pharmaceuticals; fragrances and flavors; dyes and pigments; agrichemicals; specialty monomers; functional materials; surfactants; and biologically active molecules.

28. The method of claim 27, wherein said acid-labile group protected organic molecule is a 5′-protected oligonucleotide.

29. The method of claim 21, wherein said acid-labile group protected organic molecule is protected by an acid-labile protecting group selected from the group consisting of ethers, esters, carbonates, and benzyl-type groups.

30. The method of claim 29, wherein said acid-labile protecting group is selected from the group consisting of carbonate; acetyl; t-butyl; t-butoxymethyl; methoxymethyl; tetrahydropyranyl; 1-ethoxyethyl; 1-(2-chloroethoxy)ethyl; p-chlorophenyl; 2,4-dinitrophenyl; benzyl; 2,6-dichlorobenzyl; diphenylmethyl; p-nitrobenzyl; bis(2-acetoxyethoxy)methyl (ACE); 2-trimethylsilylethyl; trimethylsilyl; triethylsilyl; t-butyldimethylsilyl; t-butyldiphenylsilyl; triphenylsilyl; [(triisopropylsilyl)oxy]methyl (TOM); benzoylformate; chloroacetyl; trichloroacetyl; trifluoroacetyl; pivaloyl; benzoyl; p-phenylbenzoyl; 9-fluorenylmethyl carbonate; mesylate; tosylate; triphenylmethyl (trityl); monomethoxytrityl (MMT); dimethoxytrityl (DMT); trimethoxytrityl; 1 (2-fluorophenyl)-4-methoxypiperidin-4-yl (FPMP); 9-phenylxanthine-9-yl (Pixyl); and 9-(p-methoxyphenyl)xanthine-9-yl (MOX) groups; and combinations of two or more of these groups.

31. The method of claim 21, wherein the temperature of the feed stream is from −78° C. to 120° C.; or wherein the beads of the acidic cation exchange catalyst are disposed in a bed, wherein the bed has a volume, and wherein the flow rate of the feed stream through the volume of the beads is 0.1 BV/hr to 240 BV/hr.
